(19) United States
(12) Patent Application Publication
(10) Pub. No.: US 2007/0087048 A1
(43) Pub. Date: Apr. 19, 2007

(54) Oral dosage combination pharmaceutical packaging

(76) Inventors: Andrew L. Abrams, Westport, CT (US); Anand V. Gumaste, West Windsor, NJ (US); Raymundo A. Sison, Princeton, NJ (US)

Correspondence Address: Norman P. Soloway;HAYES SOLOWAY P.C., Suite 140, 3450 E. Sunrise Drive, Tucson, AZ, 85718, US

(21) Appl. No.: 11/549,492

(22) Filed: Oct. 13, 2006

Related U.S. Application Data
(63) Continuation-in-part of application No. 10/479,438, filed on Dec. 1, 2003.
(60) Provisional application No. 60/294,786, filed on May 31, 2001.
(60) Provisional application No. 60/727,029, filed on Oct. 14, 2005.

(51) Int. Cl. A61K 9/64; A61K 9/48

(52) U.S. Cl. 424/451; 424/456

(57) ABSTRACT

A pharmaceutical delivery package comprising fixed unit dose quantities of two or more different active pharmaceutical ingredients (a) combined in a single delivery package, and (b) segregated from one another within said package wherein said package comprises a core containing a first active pharmaceutical ingredient surrounded at least in part by a capsule containing said second active pharmaceutical ingredient.

### CROSS REFERENCE TO RELATED APPLICATIONS

This application is a continuation-in-part of co-pending application Ser. No. 10/479,438, filed Dec. 1, 2003, which is, in turn, a 371 of PCT/US02/16185, filed May 22, 2002, which claims benefit of U.S. Provisional Application Ser. No. 60/294,786, filed May 31, 2001. This application also claims the benefit of co-pending application Ser. No. 10/756,124, filed Jan. 12, 2004 and of U.S. Provisional Application Ser. No. 60/727,029, filed Oct. 14, 2005.

### BACKGROUND OF THE INVENTION

The present invention relates to the packaging of pharmaceuticals and drugs for medical uses. The invention has particular utility in the packaging of combinations of two or more pharmaceuticals and drugs for the same or co-morbid therapy, and will be described in connection with such utility, although other utilities are contemplated.

### DESCRIPTION OF THE PRIOR ART

The convenience of co-administered two or more active pharmaceutical ingredients in a unit dosage form, as opposed to the administration of a number of separate doses of two or more pharmaceuticals at regular intervals, has been recognized in the pharmaceutical arts and is described in our prior U.S. Pat. Nos. 6,428,809 and 6,702,683, and co-pending application Ser. Nos. 10/756,124 and 10/479,438 and Provisional Application No. 60/727,029. Advantages to the patient and clinician include (1) minimization or elimination of local and/or systemic side effects; (2) more effective treatment of co-morbid conditions; (3) improved polypharmacy; and (4) better patient compliance with overall disease management, which in turn may lead to reduced costs due to fewer trips to the physician, reduced hospitalization, and improved patient well-being.

In our aforesaid U.S. Pat. Nos. 6,428,809 and 6,702,683 we have described packaging two or more active pharmaceuticals or drugs, segregated from one another, in a readily ingestible pharmaceutical delivery package which may take the form of, for example, a tablet or capsule. Various drug combinations are described and claimed in our aforesaid patents.

### SUMMARY OF THE INVENTION

The present invention provides improvements over the pharmaceutical delivery packages described in our aforesaid patents. An embodiment of the present invention provides a fixed dose combination medication delivery package which is simple to manufacture. More particularly, the embodiments of the present invention provide a pharmaceutical delivery package comprising fixed unit dose quantities of two or more different active pharmaceutical ingredients (a) combined in a single delivery package, and (b) segregated from one another within said package wherein said package comprises a core containing a first active pharmaceutical ingredient surrounded at least in part by a capsule containing a second active pharmaceutical ingredient. The active pharmaceutical ingredient is defined here as either single pharmaceutical ingredient, optionally combined with appropriate excipients, or more than one pharmaceutical ingredient, optionally combined with appropriate excipients. The present invention provides certain unique and advantageous combinations of drugs that address or overcome one of several issues relating to combinational drug therapy, including more efficient treatment of co-morbid conditions, polypharmacy, reduction of adverse side effects, adjuctive therapy and known drug interactions. In one embodiment of the invention, the delivery package is designed to provide for essentially simultaneous release of the two or more pharmaceutical ingredients. In another embodiment, the pharmaceutical delivery package provides for different release rates of the two or more pharmaceutical ingredients, or differential release of the two or more pharmaceutical ingredients. By way of example, the invention provides a combination medication delivery package that includes pharmaceutical ingredients providing combinational therapy or polypharmacy for treatment of diabetes such as diabetes and hyperlipidemia, and diabetes and hypertension. And yet another exemplary embodiment, the invention provides combinational pharmacology for treating hyperlipidemia and hypertension. In a particular embodiment, the present invention provides a package in which the active ingredients are segregated from one another by a physical barrier such that the package may be broken or split into two halves, with the active medications divided essentially equally between the halves.

As used herein the term “fixed dose combination medication delivery package” is one in which two or more drug components are packaged together, isolated from one another, in a single dosage form. The drug components may each comprise an active pharmaceutical ingredient or one of the drug components may comprise an active pharmaceutical ingredient while the other comprises a substance that effects the other ingredient, such as, through an acid base reaction, or a substance that potentiates or suppresses the other in a known and predictable manner, or a substance that suppresses or increases absorption time or uptake of the other ingredient, or a substance that suppresses or increases metabolism through enzymatic activity and effect absorption of the other ingredient. Also, in yet another embodiment, the pharmaceutical delivery package includes two or more pharmaceutical ingredients packaged in a manner whereby one or more of the ingredients will be released at different sites within the alimentary canal.

### BRIEF DESCRIPTION OF THE DRAWINGS

Further features and advantages of the present invention will become clear from the following detailed description taken in conjunction with the accompanying drawings, wherein like numerals depict like parts, and wherein:

FIGS. 1A-1H diagrammatically illustrate the formation of a combination medication delivery system in accordance with one embodiment of the present invention;

FIGS. 2A-2E diagrammatically illustrate the formation of a combination medication delivery system in accordance with a second embodiment of the present invention;

FIGS. 3A-3B diagrammatically illustrate how a combination medication delivery system of FIG. 2 may be divided or split into two half doses.

FIGS. 4A-4C diagrammatically illustrate embodiments of the combination medication delivery system according to the present invention enabling differential release of the active pharmaceutical ingredients.

FIGS. 5A-5I diagrammatically illustrate embodiments of the combination medication delivery system.

### DETAILED DESCRIPTION OF PREFERRED EMBODIMENTS

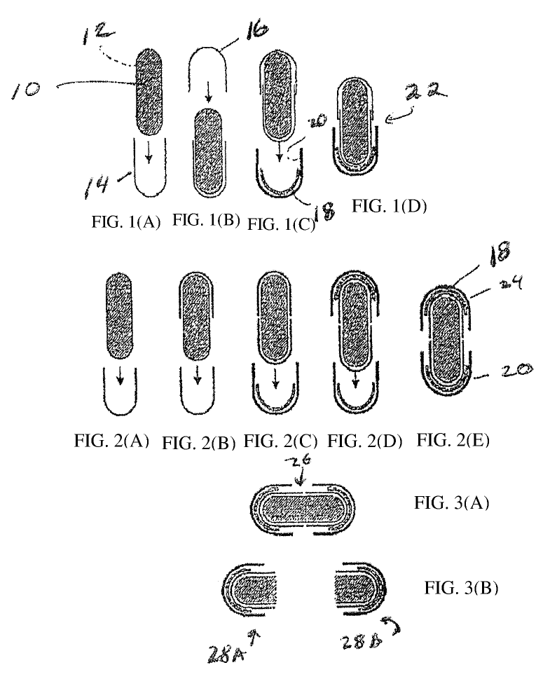

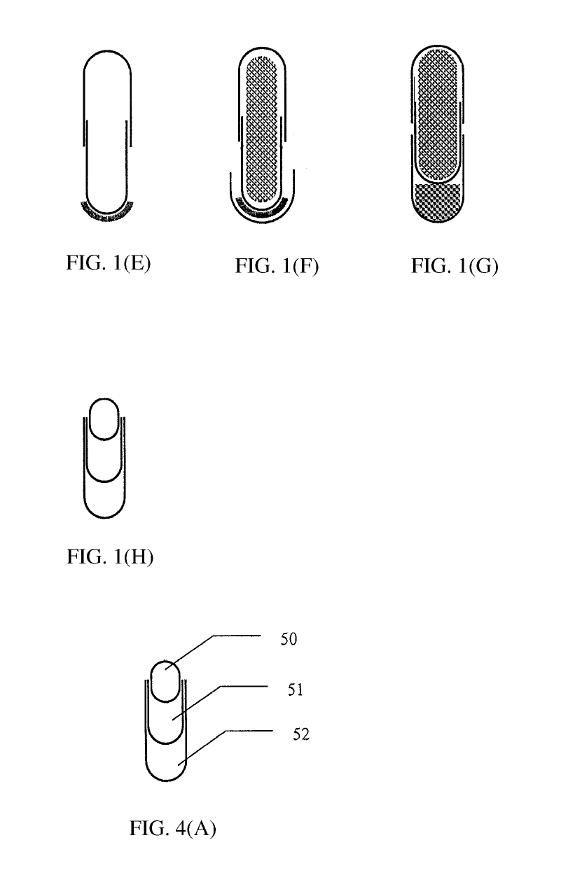

Referring first to FIGS. 1A-1B, there is diagrammatically illustrated the formation of a combination medication delivery system in accordance with one embodiment of the invention. Referring first to FIG. 1A, a core 10 comprising a controlled amount of a first pharmaceutical ingredient may be formed in a conventional manner as a tablet, capsule or caplet by combining the active pharmaceutical ingredient with a filler and binders, and shaping and forming the tablet, capsule or caplet in known manner. The core tablet, capsule or caplet 10 is then coated with a barrier material 12 such as a gelatin, a starch or a cellulose such as hydroxypropylmethylcellulose shown in phantom at 12. Alternatively, core 10 may be sealed within a two-piece capsule 14, 16 formed of, for example, gelatin such as press fit gel caps available from Capsugel, Inc. of Morris Plains, N.J..

Referring to FIGS. 1C-1D, a controlled amount of a second active pharmaceutical ingredient 18 is loaded into the bottom of a capsule half shell 20. The capsule half shell 20 which is sized to fit over the core 10 is assembled to the core 10 and fixed in place by shrink or press fitting to form a medication delivery system comprising two pharmaceuticals, indicated generally at 22.

The active pharmaceutical ingredients 10 and 18 can be in the form of a powder, including fine powder, coarse powder, or powder comprising several different fractions, as well as in the form of pellets or beads or a tablet. Additionally the active pharmaceutical ingredients 10 and 18 can be in a liquid or semi-liquid form, which can facilitate precision dosing. The liquid can be formed as a mixture of the drug and a solvent, or as a solution of the drug in a solvent, with the solvent preferably quickly evaporating or the liquid formulation quickly solidifying after dosing into the capsule or half-capsule. The liquid formulation of the drug can be additionally used to create an attachment force between the components of the combination medication delivery system in accordance with an embodiment of the present invention. Upon complete evaporation of solvent, a strong bond can be formed between the components of the combination medication delivery system such as the capsule half shell 20 and capsule part 14.

In yet another embodiment of the present invention, a powder form of the active pharmaceutical ingredient 18 is loaded into the capsule half shell 20, and a small quantity of a suitable solvent, such as water, is further dosed into the capsule half shell 20 thus creating a viscous mixture of the active pharmaceutical ingredient 18 and the solvent. Upon complete evaporation of solvent, a strong bond can be formed between the components of the combination medication delivery system such as the capsule half shell 20 and capsule part 14.

Referring now to another embodiment of the present invention shown in FIGS. 1E and 1F, a liquid formulation of the active pharmaceutical ingredient can be coated on the outside of the capsule part 14 by using dip coating, as shown in FIG. 1E and then covered with the capsule half shell 20, as shown in FIG. 1F. Upon complete evaporation of solvent, a strong bond can be formed between the components of the combination medication delivery system such as the capsule half shell 20 and capsule part 14. Referring now to FIG. 1G, another embodiment of the present invention is illustrated, wherein larger quantity of semi-solid formulation of the active pharmaceutical ingredient is placed into the capsule half shell 20.

Referring now to FIG. 1H, another embodiment of the present invention is illustrated, wherein more than 2 different active pharmaceutical ingredients are incorporated into the combination medication delivery system according to the present invention.

Alternatively, as illustrated in FIGS. 2A-2E, the second active pharmaceutical ingredient 18 may be split and loaded into two capsule half shells 20, 24 which are assembled to the core 10 and press or shrink fitted to one another. If desired, each capsule half shell 20, 24 may contain controlled amounts of different medications.

Referring now to FIGS. 3A-3B, it is illustrated how a combination medication delivery system of FIG. 2 may be divided or split into two half doses.

In another embodiment of the present invention, combinations of active pharmaceutical ingredients are incorporated into a combination medication delivery system enabling simultaneous release, differential release, and/or extended release of ingredients in the patient's alimentary canal. For simultaneous release, two or more active pharmaceutical ingredients incorporated into a combination medication delivery system are released practically simultaneously as the capsules or capsule components dissolve in the patient's alimentary canal. For differential release applications, specific capsule assemblies are enabled wherein one of the components is released before or after another component or components, using design and wall thickness and/or wall composition, including solubility in acidic and/or alkaline media. Specifically, by varying capsule wall composition, porosity capsule material curing, and wall thickness, differential release of the active pharmaceutical ingredients is achieved.

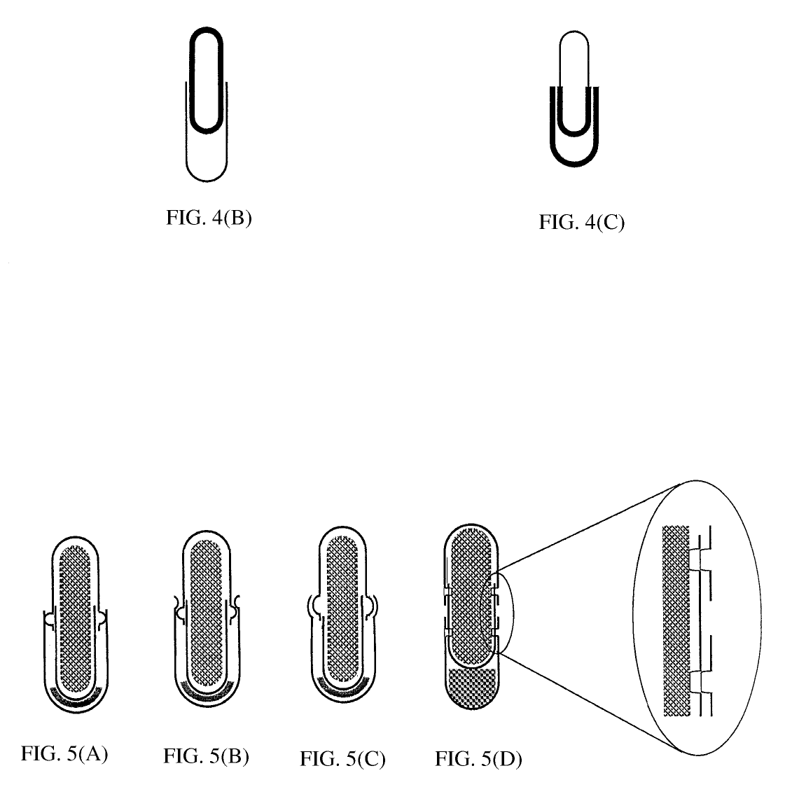

Referring now to FIG. 4A, active pharmaceutical ingredients inside compartments 50 and 52 are released first, as the capsule walls of compartments 50 and 52 are dissolving faster due to protection of the compartment 51 by the walls of the compartments 50 and 52.

Referring now to FIGS. 4B and 4C, some of the compartments of the combination medication delivery system are shown as having, for illustration purposes, a thicker wall or walls. The thicker wall as shown in these figures indicates slower dissolution rate of the wall due to higher thickness, different wall material, or both. Different material composition of the wall of the compartments shown in FIGS. 4B and 4C also can make the corresponding compartment resistant to immediate dissolution, thus delaying the release of the active pharmaceutical ingredient from the corresponding compartment. This can result in desirable late release or release in a different location along the alimentary canal. Furthermore, a compartment wall not soluble in acidic environment of the stomach can be made soluble in more neutral to alkaline environment of the small intestine, thus enabling release of the active pharmaceutical ingredient incorporated in said compartment in the small intestine. Thus one or more of several active pharmaceutical ingredients contained in the combination medication delivery system according to the present invention can be delivered to the stomach, while another active pharmaceutical ingredient or ingredients can be delivered to small intestine.

The above embodiments permit simultaneous or differential time release as well as differential spatial release of active pharmaceutical ingredients. In addition to delivery to different parts of gastrointestinal tract, combinations of active pharmaceutical ingredient with a fast action delayed action is possible, such as pain medication. Another application of the present invention is for delivery combinations wherein for example, first active pharmaceutical ingredients should be taken by the patient before food intake, while second active pharmaceutical ingredient should be taken after food intake. The combination medication delivery system taken before food intake, with delayed release of the second active pharmaceutical ingredient. Another embodiment of the present invention comprises combination medication delivery system wherein active pharmaceutical ingredients are released differentially because they can interact if released simultaneously due to chemical interactions between ingredients, changes in the pH or other parameters in the vicinity of the dissolving ingredient, or ingredients which can have a detrimental effect on the action or absorption of another ingredient.

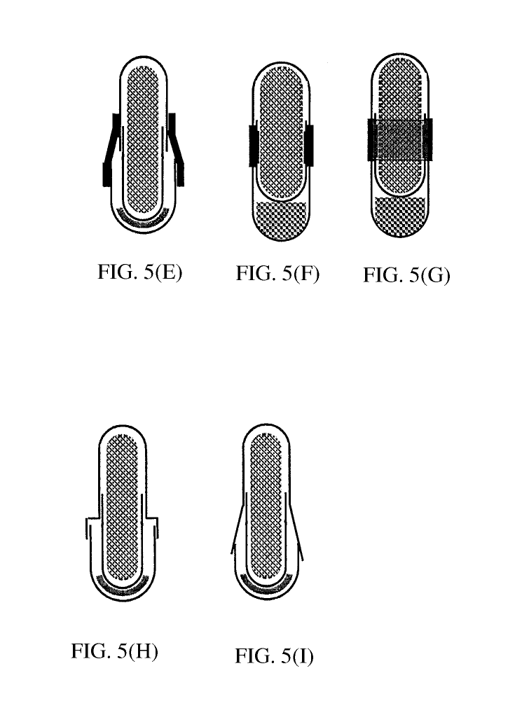

Referring now to FIGS. 5A-5I, the combination medication delivery system assembly can be further reinforced or components of the assembly joined by utilizing a tight components fit. In an embodiment, a locking ring or locking ring-groove combination, as shown in FIGS. 5A; 5B, 5C, and 5D, or a polymer band, as shown in FIGS. 5E, 5F, and 5G. In addition, a mechanism comprising a locking tight fit groove as illustrated by FIGS. 5H and 5I enables secure assembly of the combination medication delivery system of the present invention. Other methods, including forming a bond between components as was described above and depicted in FIGS. 1E and 1F above are possible. Still other methods of securing combination medication delivery system assembly are possible, including a hydroalcoholic or other liquid seal, using shrink wrap-like securing mechanism and the like. The mechanisms of securing the combination medication delivery system assembly are not limited to these described above and other mechanisms are also possible.

As discussed in our aforesaid parent patents and patent applications, there are many combinations of drugs that advantageously may be employed for treatment of co-morbid diseases, polypharmacy and/or reduce side effects of treatment. By way of example, eighty plus percent of diabetics reportedly are also hypertensive. Hyperlipidemia also is frequently concurrent with diabetes. Thus, an anti-diabetic agent conventionally used for treating diabetes such as a sulfonylurea, a meglitinide, a biguanide, an insulin sensitizer such as thiazolidinedione, or an alpha-glucosidase inhibitor may be combined with a drug useful for treating hypertension or hyperlipidemia. For example, a dose of sulfonylurea (e.g., Glipizide) can be combined in a single delivery system with a dose of a statin (e.g., Atorvastatin), a fibrate, a bile acid sequestrant (e.g., Cholestipol), a cholesterol absorption inhibitor or niacin. Likewise, a sulfonylurea can be combined with a bile acid sequestrant. Similarly, a drug for treating diabetes may be combined with an ACE inhibitor, an angiotension II antagonist, a calcium blocker, a beta-blocker, or a diuretic. An example is a combination of a biguanide (e.g., Metformin) coadministered with a calcium channel blocker (e.g., Amlodipine). Another example would be the combination of a meglitinide (e.g., Repaglinide) and an angiotension II antagonist (e.g., Losartan). Also, drug combinations may be selected based on the following criteria:

The possibility of a pharmacodynamic interaction. Drug combinations may be selected which exhibit affinity for the same receptors or may produce similar effects on physiologic function, related or not to their mechanism of action.

The possibility of a pharmacokinetic interaction. A pharmacokinetic interaction can manifest in several ways, some of which can be monitored in vivo and some of which cannot. One drug product may be selected based on its ability to alter the absorption or excretion of another product, change its distribution into one or more tissues, or change its pattern or rate of metabolism. Drugs may compete for serum protein binding, resulting in an increase in circulating free levels and tissue uptake of one drug.

The possibility of a toxicologic interaction (e.g., where the target organs for toxicity are similar for each drug). A possible lowering of a previously determined no-effect dose for one or both drug products and/or more severe toxicities in the affected organs should be considered, where applicable.

The margin of safety for each drug product. If one or more of the drugs has a narrow margin of safety (i.e., causes serious toxicity at exposures close to the predicted clinical exposure), then the possibility of drug interaction needs to be considered.

The possibility that the drugs compete for or alter the activity or endogenous levels of the same enzymes or other intracellular molecules should be considered (e.g., co-administration of two prooxidants could deplete endogenous levels of glutathione).

The possibility of a chemical interaction. One drug may chemically modify another drug (e.g., one drug may oxidize, methylate, or ethylate the other drug). This could result in new molecular entities with new toxicities. However, this effect can largely be avoided by providing for delayed release of one of the drugs.

The possibility that one drug may compromise the effectiveness of another drug.

Various embodiments of the invention will now be further described with reference to the following non-limiting examples:

(1) Combination #1: Enalapril maleate¹ and analogs and isomers thereof are ACE inhibitors used for the treatment of hypertension. This drug may be used with the following and analogs and isomers of beta adrenergic-blocking agents, methyldopa, nitrate, calcium blocking agents, Hydralazine⁶, Prazosin⁷ and Digoxin⁸ without clinically significant side effects. One or more of these agents may be packaged as above described with a drug for treatment of diabetes such as a sulfonylurea, a meglitimide, a biguanide, an insulin sensitizer or an alpha-glucosidase inhibitor.

(2) Combination #2: A hypoglycermic agent such as Metformin HCl² and analogs and isomers thereof may be packaged as above described with an angiotensin converting enzyme inhibitor (ACE inhibitor).

(3) Combination #3: A diabetes drug as above described in Combination #1 or #2 may be packaged as above described with an angiotensin II receptor antagonist such as Losartan potassium³ and/or Valsartan⁴.

(4) Combination #4: A diabetes drug as above described may be packaged as above described with a Beta Adrenergic Blocking Agent such as Bioprolol fumarate⁵ or Metoprolol succinate⁶.

(5) Combination #5: A diabetes drug as above described may be packaged as described in Combinations #1 or #2 may be packaged with a Calcium Channel Blocking Agent such as Amlodipine⁷ or Nifedipine⁸.

(6) Combination #6: A diabetes drug as above described may be packaged with a Periferal Adrenergic Blocking Agent such as Prazosin hydrochloride⁹.

(7) Combination #7: A diabetes drug as above described may be packaged with an Adrenergic central stimulant such as Methyldopa¹⁰ or Clonidine¹¹.

(8) Combination #8: A biguanide such as Metformin¹⁴ may be packaged as above described with a sulfonylurea such as Glipizide¹⁵.

(9) Combination #9: A biguanide such as Metformin¹⁴ may be packaged as above described with a thiazolidinedione such as rosiglitazone maleate¹⁶.

(10) Combination #10: A biguanide such as Metformin¹⁴ may be packaged as above described with an alpha glucosidase inhibitor such as Cerivastatin¹⁷.

(11) Combination #11: A short acting oral insulin may be packaged as above described with sustained release oral insulin.

The drug delivery system of the present invention also allows three drug combinations such as diabetes drugs and ACE Inhibitors combined with Beta Blockers, methyldopa nitrates, calcium channel blockers, Hydralazine¹², Prazosin¹³, Digoxin¹⁴ as well as multiple combinations of drugs.

(12) Combination #12: A diabetes drug may be packaged with an ACE Inhibitor and a Beta Blocker.

(13) Combination #13: A diabetes drug such as described in Combinations #1 or #2 may be packaged with a HMG-CoA reductase inhibitor such as Simvastatin³⁵, Atorvastatin³⁶, or Pravastatin³⁷, and with a bile acid sequestrant such as Colestipol hydrohloride³⁸.

(14) Combination #14: A diabetes drug such as described in Combinations #1 or #2 may be packaged with a HMG-CoA reductase inhibitor and with a niacin compound.

(15) Combination #15: A diabetes drug such as described in Combinations #1 or #2 may be packaged with a HMG-CoA reductase inhibitor or Combination #14, and with a hypolipidemia agent such as Gemfibrozil³⁹.

While the above embodiments of the invention has been described with particular drug combinations segregated from one another, it will be understood that some of the above-listed drug combinations also may be blended and packaged in a single tablet, capsule or caplet when chemical interaction is not a problem.

Other embodiments of the present invention are directed towards combinations of at least one active pharmaceutical ingredient and at least one substance which can be an active pharmaceutical ingredient or non-pharmaceutical ingredient and which is mitigating the negative effects of said first active pharmaceutical ingredient, or promoting/enhancing action of said first active pharmaceutical ingredient, or is promoting general health and well-being of the patient taking said first active pharmaceutical ingredient. The following non-limiting examples are illustrating this aspect of the embodiments of the present invention:

Example 16: A combination of first active pharmaceutical ingredient which may cause a side effect with a second active pharmaceutical ingredient medication mitigating side effect of the first active pharmaceutical ingredient are combined in a single delivery package. Examples include first active pharmaceutical ingredient with side effect causing, e.g., constipation, nausea, gas/bloating, heartburn, pain or cramps; and a second active pharmaceutical ingredient, mitigating the above side effect of the first ingredient, e.g. correspondingly laxative medication, nausea treatment medication, anti-gas and anti-bloating medication, anti-acid medication, pain reliever & muscle relaxant medication. More specific example may include pain medication causing constipation and nausea, e.g. oral narcotic with the second ingredient containing stool softener and anti-nausea components.

Example 17. In another embodiment of the present invention, a first active pharmaceutical ingredient is combined with a second active pharmaceutical ingredient which controls and stops the action of the first ingredient after the time necessary for the action of the first ingredient. As an example, a combination of anti-cancer drug such as Methetrexate with immediate release, and the “quencher” substance, such as L-leukovorin, with delayed release, can be advantageously delivered within the combination medication delivery system.

Example 18: In another embodiment of the present invention, a first active pharmaceutical ingredient is combined with a second active pharmaceutical ingredient or a substance which optimizes the pH in the immediate vicinity of the first active pharmaceutical ingredient for facilitating dissolution, and/or absorption of the first active pharmaceutical ingredient. Additionally, control and/or neutralization of the stomach acid to slow down first active pharmaceutical ingredient breakdown can be affected thus improving the bioavailability of the first active pharmaceutical ingredient. Non-limiting examples of pH controlling substances include pH buffering compounds known in the art.

Example 19: In another embodiment of the present invention, a first active pharmaceutical ingredient which is fat soluble is combined with a second active pharmaceutical ingredient or a substance containing oil for better drug solubility and absorption.

Example 20: In another embodiment of the present invention, a first active pharmaceutical ingredient is combined with an enzyme wherein said enzyme facilitates active pharmaceutical ingredient absorption and/or bio-availability or mitigates side effects.

Example 21: In another embodiment of the present invention, a first active pharmaceutical ingredient is combined with a nutraceutical or a vitamin. Non-limiting examples include combination of (i) Nexium (esomeprazole) which changes the pH in the stomach and thus prevents absorption of B12 vitamin which can only happen at low pH, with B-group vitamins and (ii) Anti-viral active pharmaceutical ingredients with vitamin C or multivitamin supplements.

Example 22: In another embodiment of the present invention, a first active pharmaceutical ingredient is combined with a surfactant which facilitates absorption or vice versa, inhibits absorption in the certain part of the alimentary canal.

Example 23: In another embodiment of the present invention, a first active pharmaceutical ingredient is combined with a sleeping aid.

Another embodiment of the present invention is directed towards combinations of at least two active pharmaceutical ingredients within the same class of pharmaceuticals treating or preventing the same symptoms or same disease (polypharmacy), such as infectious disease, metabolic disorders, cardiovascular disease, pain, cancer, transplant-related treatment, gastrointestinal disorders, respiratory diseases, autoimmune diseases, vaccines, etc. The following non-limiting examples are illustrating this embodiment of the present invention:

Example 24: Combination of anti-infective active pharmaceutical ingredients, with examples including at least two antibiotics combined, resulting in a broad spectrum anti-bacterial action. Another example includes a combination of anti-viral and anti-bacterial pharmaceutical ingredients resulting in a treatment of an infection with unknown pathogen as well as treatment of bacterial infections often following viral infections. Yet another example includes a combination of at least two active pharmaceutical ingredients which are treating cancer or managing the symptoms of cancer, for example topoisomerase inhibitor drug and anti-cancer monoclonal antibody drug. Another example includes a combination of antibiotic with antibiotic potentiators. Potentiators confer increased activity to pharmaceutical agents, such as, for instance, antibiotics. Although potentiators may lack themselves any antibacterial activity, in combination with antibiotics, such as for example, erythromycin, chloramphenicol, tetracycline, linezolid, clindamycin or rifampin, potentiators promote and significantly increase the activity of the pharmaceutical agent, in this example, antibiotic.

Example 25: In another embodiment of the present invention, the same active pharmaceutical ingredient is combined in at least two formulations, including a fast release or fast action and a slow release or long term action formulation. The slow release or long term action can be achieved by differential release capsule components design, as discussed above, or by formulation of the drug, excipients and tablet forming means, and other means available to these skilled in the art, with beneficial effects including better treatment or relief of symptoms and potential for the decrease of the overall medication intake. Specific non-limiting examples include: nitroglycerin, with fast acting/fast dissolving formulation providing for a fast action for acute treatment with a slow release formulation for maintenance; antibiotic with fast action/fast dissolution formulation for immediate increase of the concentration in blood plus slow release; pain medication, with a fast acting formulation for immediate pain relief help combined with a slow release pain maintenance medication; sleeping aid with a fast dissolving or fast acting formulation for immediate effect combined with a delayed release for maintenance throughout the night, with specific non-limiting example including Ambien.

Example 26: In another embodiment of the present invention, at least two anti-cholesterol pharmaceutical ingredients such as statins of different types are combined in the combination medication delivery system. Since effects of statins are highly individual, a combination medication is advantageous.

Example 27: In another embodiment of the present invention, a broad spectrum anti-hypertensive combination comprises two or more hypertension-reducing drugs in the combination medication delivery system, including medications of the same type, such as beta-blockers or diuretics, or medications of different types or classes, such as beta-blocker and diuretic.

Various other changes may be made without departing from the spirit and scope of the invention. For example, the above-described capsules may be used with various drug combinations as described in our earlier U.S. Pat. Nos. 6,428,809 and 6,702,783, and the drug combinations described in our co-pending application Ser. Nos. 10/756,124 and 10/479,438. Still other drug combinations, which term may also include vitamins, dietary supplements, minerals and nutraceuticals, which may be used with the above-described capsules or with the combination capsules, tablets or caplets described in our earlier patents and pending applications, include combination drug therapies for treating infectious disease, e.g., AIDS, TB and malaria, and for pain management, e.g., nonsteroidal anti-inflammatory drugs/proton pump inhibitors (NSAIDS/PPI). These include, by way of example, and not limitation:

Example 28. In another embodiment of the present invention, at least two anti-malaria drugs are combined in the combination medication delivery system. Specific Examples of potential drug combinations include, Artesunate and Mefloquine; Artemether and Lumefantrine; Chloroquine and Paracetamol. More generally, a combination of at least two of the following representative anti-malaria drugs in the combination medication delivery system are exemplified: Artemether; Lumefantrine; Artensunate; Amodiaquine HCl; Atovaquone-proguanil; Quinine Sulfate; Chloroquine Sulfate; Hydroxychloroquine Sulfate; Doxycycline; Mefloquine; Primaquine; Sulfadoxine; Pyrimethamine; Paracetamol.

Example 29. In another embodiment of the present invention, at least two HIV treatment medications are combined in the combination medication delivery system. Specific Examples of potential drug combinations include, at least two of the nucleoside reverse transcriptase inhibitor (NRTI) medications, including e.g. Abacavir; lamivudine; Didanosine; Emtricitabine; Stavudine; Tenofovir. Another example includes combining a non-nucleoside reverse transcriptase inhibitor (NNRTI) and a nucleoside reverse transcriptase inhibitor (NRTI) e.g. Nevirapine (NNRTI) and didanozine (NRTI); Efavirenz (NNRTI) and abacavir sulfate (NRTI). Yet another example includes combining two NRTI's and one NNRTI e.g. Abacavir and lamivudine and efavirenz or Abacavir and lamivudine and nevirapine. Still another Example includes combining at least two 2 NRTI's and a PPI: Abacavir and lamivudine and lopinavir/ritonavir. Still another example includes a combination of at least two of the anti-HIV drugs selected from the group comprising: abacavir sulfate; didanozine; stavudine; tenofovir; disoproxil; fumarate; zidovudine; lamivudine; emtricitabine; lopinavir/ritonavir; nevirapine; efavirenz; nelfinavir. Still other combinations include combination of AZT and 3TC; combination of abacavir and AZT and 3TC; a combination of lopinavir and ritonavir; combinations of ABC and 3TC; and combination of emtricitabine and tenofovir.

Example 30. In another embodiment of the present invention, at least two of Tuberculosis treatment medications are combined in the combination medication delivery system. Specific Examples of potential combinations include at least two of the following medications: Isoniazid; Rifampicin; Pyrazinamide; Ethambutol HCl; Streptomycin; Capreomycin; Cycloserine; Protionamide; Macrolides; Fluoroquinolones; p-Salicylic acid.

Example 31. In another embodiment of the present invention, at least two of the pain treatment medications are combined in the combination medication delivery system. Specific Examples of potential combinations include at least two of the following medications: Aspirin; Carbex; Codeine; Luvox; Marplan; Nardil; Neurotin; OxyContin; Pamate; Topamax; Tylenol/Acetaminophen; Vicodin; Xyrem; Zarontin; Zoloft; Zomig.

Example 32. Another embodiment of the present invention is a combination of aspirin or acetylsalicylic acid combined in the combination medication delivery system with a active ingredient mitigating side effects of aspirin, such as effects related to the acidity of aspirin. Specific Examples of potential combinations include buffering compounds and anti-acid compounds in combination with aspirin.

Example 33. Another embodiment of the present invention is a combination therapy for treatment of lupus nephritis. Specific example includes combination of methylprednisolone and cyclophosphamide.

Still other changes are permissible. For example, a pre-formed tablet, capsule or caplet containing one pharmaceutical ingredient may be obtained from the manufacturer. Then, a compounding pharmacist may encase that pre-formed tablet within an outer capsule in which a second pharmaceutical ingredient is loaded. This permits a compounding pharmacist to produce custom drug combination packages. Also, if desired, the pharmaceutical delivery system may be scored adjacent its mid-point 26 so that the delivery system may be broken into two equal halves 28A, 28B, so that the user may create half dose tablets each half containing equal amounts of both medications. (See FIGS. 3A-3B).

Various other changes may be possible without departing from the spirit and scope of the invention. For example, the core may comprise a capsule containing a liquid or gel. Still other changes are possible.

1. A pharmaceutical delivery package comprising fixed unit dose quantities of two or more different pharmaceutical ingredients (a) combined in a single delivery package, and (b) segregated from one another within said package wherein said package comprises a core containing a first pharmaceutical ingredient surrounded at least in part by a capsule containing a second pharmaceutical ingredient.

2. A pharmaceutical delivery package according to claim 1, wherein said capsule comprises a half capsule.

3. A pharmaceutical delivery package according to claim 1, wherein said capsule comprises a two piece capsule.

4. A pharmaceutical delivery package according to claim 3, comprising a first pharmaceutical ingredient contained in the core, a second pharmaceutical ingredient contained in one of said capsule pieces, and a third pharmaceutical ingredient contained in the other of said capsule pieces.

5. A pharmaceutical delivery package according to claim 1, wherein the core is coated with a barrier material.

6. A pharmaceutical delivery package according to claim 5, wherein the barrier material is selected from the group consisting of a gelatin, a starch and a cellulose material.

7. A pharmaceutical delivery package according to claim 6, wherein the cellulose material comprises hydroxypropylmethylcelluose.

8. A pharmaceutical delivery package according to claim 3, wherein said capsule pieces are joined together by shrink or press fitting.

9. A pharmaceutical delivery package according to claim 1, wherein at least one of the two or more different pharmaceutical ingredients is in a powder, pellet or bead form.

10. A pharmaceutical delivery package according to claim 1, wherein at least one of said pharmaceutical ingredients is in a liquid, or semi-liquid or gel form.

11. A pharmaceutical delivery package according to claim 1, wherein the capsule is bonded to the core.

12. A pharmaceutical delivery package according to claim 3, wherein the capsule pieces are bonded to one another.

13. A pharmaceutical delivery package according to claim 3, wherein the capsule pieces are joined together by mating rings, a locking groove and ring, or a locking band.

14. A pharmaceutical delivery package according to claim 3, wherein the capsule pieces are joined together by a set in place liquid.

15. A pharmaceutical delivery package according to claim 1, wherein the capsule walls have a physical property selected from thickness, composition, solubility and porosity whereby release of active pharmaceutical ingredients contained therein into the alimentary canal may be controlled.

16. A pharmaceutical delivery package according to claim 15, wherein the capsule walls are acid resistant, and are permeable or soluble in a neutral to alkaline environment.

17. A pharmaceutical delivery package according to claim 1, wherein the core comprises a capsule containing a liquid or gel.

18. A pharmaceutical delivery package according to claim 1, wherein one of the pharmaceutical ingredients is selected from the group consisting of a vitamin, a dietary supplement, a mineral and a nutraceutical.

19. A pharmaceutical delivery package according to claim 1, comprising combinations of pharmaceutical ingredients selected from the group consisting of an anti-diabetic agent and an anti-hypertensive agent; an anti-diabetic agent and anti-hyperlipidemia agent, wherein the anti-diabetic agent preferably is selected from the group consisting of a sulfonylurea, a meglitinide, a biguanide, an insulin sensitizer and an alpha-glucosidase inhibitor, and the anti-hypertensive agent preferably is selected from the group consisting of an ACE inhibitor, an angiotension II antagonist, a calcium blocker, a beta-blocker and a diuretic, or wherein the anti-diabetic agent preferably is selected from the group consisting of a sulfonylurea, a meglitinide, a biguanide, an insulin sensitizer and an alpha-glucosidase inhibitor, and the anti-hyperlipidemia agent preferably is selected from the group consisting of a statin, a fibrate, a bile acid sequestrant, a cholesterol absorption inhibitor and niacin.

20. A pharmaceutical delivery package according to claim 1, wherein one of the pharmaceutical ingredients is selected from an ingredient which mitigates a side effect of the other pharmaceutical ingredient; or which acts as a time control quencher for the other pharmaceutical ingredient; or which facilitates dissolution and/or absorption of the other pharmaceutical ingredient, e.g., through pH control; or, which is fat soluble and the other pharmaceutical ingredient contains a fat or oil; or which contains an enzyme for facilitating absorption and/or bio-availability of the other pharmaceutical ingredient, or mitigating side effects of the other pharmaceutical ingredient; or, which includes a surfactant which facilitates absorption or inhibits absorption in a selected part of the alimentary canal; or which comprises a sleeping aid.

21. A pharmaceutical delivery package according to claim 1, wherein the first and the second pharmaceutical ingredients are effective for treating the same symptom or disease; or the first and the second pharmaceutical ingredients are both antibiotics; or one of the pharmaceutical ingredients is an anti-viral agent, and the other pharmaceutical ingredient is an anti-bacterial agent; or one of the pharmaceutical ingredients is an antibiotic, and the other pharmaceutical ingredient is an antibiotic potentiator; or one of the pharmaceutical ingredients comprises an NRTI and the other pharmaceutical ingredient comprises an NNRTI; or one of the pharmaceutical ingredients comprises a PPI, and the other pharmaceutical ingredient comprises an NNRTI or one of the pharmaceutical ingredients comprises an NSAID, and the other pharmaceutical ingredient comprises a PPI.

22. A pharmaceutical delivery package according to claim 1, wherein the pharmaceutical ingredients comprise agents for treating infectious disease or pain.

23. A pharmaceutical delivery package according to claim 22, wherein the infectious disease comprises HIV/AIDS, TB or malaria.

24. A pharmaceutical delivery package according to claim 1, comprising two or more pharmaceutical ingredients selected from the group consisting of Enalapril maleate and analogs and isomers thereof and analogs and isomers of beta adrenergic-blocking agents, methyldopa, nitrate, calcium blocking agents, Hydralazine, Prazosin and Digoxin; a hypoglycermic agent such as Metformin HCl and analogs and isomers thereof and an angiotensin converting enzyme inhibitor (ACE inhibitor); a diabetes drug and an angiotensin II receptor antagonist such as Losartan potassium and/or Valsartan; a diabetes drug and a Beta Adrenergic Blocking Agent such as Bioprolol fumarate or Metoprolol succinate; a diabetes drug and a Calcium Channel Blocking Agent such as Amlodipine or Nifedipine; a diabetes drug and a Periferal Adrenergic Blocking Agent such as Prazosin hydrochloride; a diabetes drug and a Adrenergic central stimulant such as Methyldopa or Clonidine; a biguanide such as Metformin and a sulfonylurea such as Glipizide; a biguanide such as Metformin and a thiazolidinedione such as rosiglitazone maleate; a biguanide such as Metformin and an alpha glucosidase inhibitor such as Cerivastatin; a short acting oral insulin and a sustained release oral insulin; a diabetes drug and an ACE Inhibitor combined with a Beta Blocker, a methyldopa nitrate, a calcium channel blocker, Hydralazine, Prazosin, or Digoxin; a diabetes drug and an ACE Inhibitor and a Beta Blocker; a diabetes drug and a HMG-CoA reductase inhibitor such as Simvastatin, Atorvastatin, or Pravastatin, and with a bile acid sequestrant such as Colestipol hydrohloride; a diabetes drug and a HMG-CoA reductase inhibitor and a niacin compound; a diabetes drug and a HMG-CoA reductase inhibitor or Combination, and with a hypolipidemia agent such as Gemfibrozil; a pharmaceutical ingredient with side effect causing, constipation, nausea, gasibloating, heartburn, pain or cramp and a second pharmaceutical ingredient, mitigating the above side effect of the first ingredient, e.g. correspondingly laxative medication, nausea treatment medication, anti-gas and anti-bloating medication, anti-acid medication, pain reliever & muscle relaxant medication; a pain medication causing constipation and nausea, oral narcotic and the second ingredient containing a stool softener and/or an anti-nausea components; an anti-cancer drug such as Methetrexate with immediate release, and a “quencher” substance, such as L-leukovorin, with delayed release; a first pharmaceutical ingredient and a second pharmaceutical ingredient or a substance which optimizes or controls pH such as a buffer for facilitating dissolution, and/or absorption of the first active pharmaceutical ingredient; a first pharmaceutical ingredient which is fat soluble and a second pharmaceutical ingredient or a substance containing oil; a first pharmaceutical ingredient and an enzyme wherein said enzyme facilitates active pharmaceutical ingredient absorption and/or bio-availability or mitigates side effects; a first pharmaceutical ingredient and a nutraceutical or a vitamin, such as Nexium (esomeprazole) and B-group vitamins, and Anti-viral active pharmaceutical ingredients and vitamin C or multivitamin supplements; a pharmaceutical ingredient and a surfactant which facilitates absorption or vice versa, inhibits absorption in the certain part of the alimentary canal; a pharmaceutical ingredient and a sleeping aid; a first and second ingredient within the same class of pharmaceuticals for treating or preventing the same symptoms or same disease (polypharmacy), such as infectious disease, metabolic disorders, cardiovascular disease, pain, cancer, transplant-related treatment, gastrointestinal disorders, respiratory diseases, autoimmune diseases and vaccines; anti-infective active pharmaceutical ingredient comprising first and second antibiotics; an anti-viral and an anti-bacterial pharmaceutical ingredient; a pharmaceutical ingredient, for treating cancer and managing symptoms of cancer, for example topoisomerase inhibitor drug, and an anti-cancer monoclonal antibody drug, an antibiotic and an antibiotic potentiator; a fast release or fast action and slow release or long term action formulations of the same pharmaceutical, such as nitroglycerin, with fast acting/fast dissolving formulation providing for a fast action for acute treatment with a slow release formulation for maintenance; antibiotic with fast action/fast dissolution formulation for immediate increase of the concentration in blood plus slow release; pain medication, with a fast acting formulation for immediate pain relief help combined with a slow release pain maintenance medication; sleeping aid with a fast dissolving or fast acting formulation for immediate effect combined with a delayed release for maintenance throughout the night, such as Ambien; two anti-cholesterol pharmaceutical ingredients such as statins of different types combined in the combination medication delivery system; a broad spectrum anti-hypertensive combination comprising two or more hypertension-reducing drugs, including medications of the same type, such as beta-blockers or diuretics, or medications of different types or classes, such as beta-blocker and diuretic; two or more anti-malaria drugs such as Artesunate and Mefloquine; Artemether and Lumefantrine; Chloroquine and Paracetamol; and at least two of the following: Artemether; Lumefantrine; Artensunate; Amodiaquine HCl; Atovaquone-proguanil; Quinine Sulfate; Chloroquine Sulfate; Hydroxychloroquine Sulfate; Doxycycline; Mefloquine; Primaquine; Sulfadoxine; Pyrimethamine; Paracetamol; at least two nucleoside reverse transcriptase inhibitor (NRTI) medications, including e.g. Abacavir; lamivudine; Didanosine; Emtricitabine; Stavudine; Tenofovir, a non-nucleoside reverse transcriptase inhibitor (NNRTI) and a nucleoside reverse transcriptase inhibitor (NRTI) e.g. Nevirapine (NNRTI) and didanozine (NRTI); Efavirenz (NNRTI) and abacavir sulfate (NRTI); two NRTI's and one NNRTI, e.g. Abacavir and lamivudine and efavirenz or Abacavir and lamivudine and nevirapine; at least two 2 NRTI's and a PPI such as Abacavir and lamivudine and lopinavir/ritonavir; at least two anti-HIV drugs selected from the group consisting of: abacavir sulfate; didanozine; stavudine; tenofovir; disoproxil; fumarate; zidovudine; lamivudine; emtricitabine; lopinavir/ritonavir; nevirapine; efavirenz and nelfinavir; a combination of AZT and 3TC; a combination of abacavir and AZT and 3TC; a combination of lopinavir and ritonavir; combinations of ABC and 3TC; a combination of emtricitabine and tenofovir; at least two of Tuberculosis treatment medications selected from: Isoniazid; Rifampicin; Pyrazinamide; Ethambutol HCl; Streptomycin; Capreomycin; Cycloserine; Protionamide; Macrolides; Fluoroquinolones; and p-Salicylic acid; at least two of the pain treatment medications selected from: Aspirin; Carbex; Codeine; Luvox; Marplan; Nardil; Neurotin; OxyContin; Parnate; Topamax; Tylenol/Acetaminophen; Vicodin; Xyrem; Zarontin; Zoloft and Zomig; a pH buffering compound and/or an anti-acid compound in combination with aspirin; and a combination therapy for treatment of lupus nephritis, such as methylprednisolone and cyclophosphamide.

25. A pharmaceutical delivery package as claimed in claim 1, comprising fixed unit dose quantities of two or more different pharmaceutical ingredients (a) combined in a single delivery package, and (b) segregated from one another within said package, wherein the package, when split in half will contain equal amounts of the pharmaceutical ingredients.

26. A pharmaceutical delivery package comprising fixed unit dose quantities of two or more different active pharmaceutical ingredients (a) combined in a single delivery package, and (b) segregated from one another within said package, characterized by one or more of the following features:
(a) wherein one of the pharmaceutical ingredients is selected from the group consisting of a vitamin, a dietary supplement, a mineral and a nutraceutical;
(b) comprising combinations of pharmaceutical ingredients selected from the group consisting of an anti-diabetic agent and an anti-hypertensive agent; an anti-diabetic agent and anti-hyperlipidemia agent, wherein the anti-diabetic agent preferably is selected from the group consisting of a sulfonylurea, a meglitinide, a biguanide, an insulin sensitizer and an alpha-glucosidase inhibitor, and the anti-hypertensive agent preferably is selected from the group consisting of an ACE inhibitor, an angiotension II antagonist, a calcium blocker, a beta-blocker and a diuretic, or wherein the anti-diabetic agent preferably is selected from the group consisting of a sulfonylurea, a meglitinide, a biguanide, an insulin sensitizer and an alpha-glucosidase inhibitor, and the anti-hyperlipidemia agent preferably is selected from the group consisting of a statin, a fibrate, a bile acid sequestrant, a cholesterol absorption inhibitor and niacin;
(c) wherein one of the pharmaceutical ingredients is selected from an ingredient which mitigates a side effect of the other pharmaceutical ingredient; or which acts as a time control quencher for the other pharmaceutical ingredient; or which facilitates dissolution and/or absorption of the other pharmaceutical ingredient, e.g., through pH control; or, which is fat soluble and the other pharmaceutical ingredient contains a fat or oil; or which contains an enzyme for facilitating absorption and/or bio-availability of the other pharmaceutical ingredient, or mitigating side effects of the other pharmaceutical ingredient; or, which includes a surfactant which facilitates absorption or inhibits absorption in a selected part of the alimentary canal; or which comprises a sleeping aid;
(d) wherein the first and the second pharmaceutical ingredients are effective for treating the same symptom or disease; or the first and the second pharmaceutical ingredients are both antibiotics; or one of the pharmaceutical ingredients is an anti-viral agent, and the other pharmaceutical ingredient is an anti-bacterial agent; or one of the pharmaceutical ingredients is an antibiotic, and the other pharmaceutical ingredient is an antibiotic potentiator; or one of the pharmaceutical ingredients comprises an NRTI and the other pharmaceutical ingredient comprises an NNRTI; or one of the pharmaceutical ingredients comprises a PPI, and the other pharmaceutical ingredient comprises an NNRTI or one of the pharmaceutical ingredients comprises an NSAID, and the other pharmaceutical ingredient comprises a PPI;
(e) wherein the pharmaceutical ingredients comprise agents for treating infectious disease or pain;
(f) wherein the infectious disease comprises HIV/AIDS, TB or malaria; and
(g) comprising two or more pharmaceutical ingredients selected from the group consisting of Enalapril maleate and analogs and isomers thereof and analogs and isomers of beta adrenergic-blocking agents, methyldopa, nitrate, calcium blocking agents, Hydralazine, Prazosin and Digoxin; a hypoglycermic agent such as Metformin HCl and analogs and isomers thereof and an angiotensin converting enzyme inhibitor (ACE inhibitor); a diabetes drug and an angiotensin II receptor antagonist such as Losartan potassium and/or Valsartan; a diabetes drug and a Beta Adrenergic Blocking Agent such as Bioprolol fumarate or Metoprolol succinate; a diabetes drug and a Calcium Channel Blocking Agent such as Amlodipine or Nifedipine; a diabetes drug and a Periferal Adrenergic Blocking Agent such as Prazosin hydrochloride; a diabetes drug and a Adrenergic central stimulant such as Methyldopa or Clonidine; a biguanide such as Metformin and a sulfonylurea such as Glipizide; a biguanide such as Metformin and a thiazolidinedione such as rosiglitazone maleate; a biguanide such as Metformin and an alpha glucosidase inhibitor such as Cerivastatin; a short acting oral insulin and a sustained release oral insulin; a diabetes drug and an ACE Inhibitor combined with a Beta Blocker, a methyldopa nitrate, a calcium channel blocker, Hydralazine, Prazosin, or Digoxin; a diabetes drug and an ACE Inhibitor and a Beta Blocker; a diabetes drug and a HMG-CoA reductase inhibitor such as Simvastatin, Atorvastatin, or Pravastatin, and with a bile acid sequestrant such as Colestipol hydrohloride; a diabetes drug and a HMG-CoA reductase inhibitor and a niacin compound; a diabetes drug and a HMG-CoA reductase inhibitor or Combination, and with a hypolipidemia agent such as Gemfibrozil; a pharmaceutical ingredient with side effect causing, constipation, nausea, gas/bloating, heartburn, pain or cramp and a second pharmaceutical ingredient, mitigating the above side effect of the first ingredient, e.g. correspondingly laxative medication, nausea treatment medication, anti-gas and anti-bloating medication, anti-acid medication, pain reliever & muscle relaxant medication; a pain medication causing constipation and nausea, oral narcotic and the second ingredient containing a stool softener and/or an anti-nausea components; an anti-cancer drug such as Methetrexate with immediate release, and a “quencher” substance, such as L-leukovorin, with delayed release; a first pharmaceutical ingredient and a second pharmaceutical ingredient or a substance which optimizes or controls pH such as a buffer for facilitating dissolution, and/or absorption of the first active pharmaceutical ingredient; a first pharmaceutical ingredient which is fat soluble and a second pharmaceutical ingredient or a substance containing oil; a first pharmaceutical ingredient and an enzyme wherein said enzyme facilitates active pharmaceutical ingredient absorption and/or bio-availability or mitigates side effects; a first pharmaceutical ingredient and a nutraceutical or a vitamin, such as Nexium (esomeprazole) and B-group vitamins, and Anti-viral active pharmaceutical ingredients andvitamin C or multivitamin supplements; a pharmaceutical ingredient and a surfactant which facilitates absorption or vice versa, inhibits absorption in the certain part of the alimentary canal; a pharmaceutical ingredient and a sleeping aid; a first and second ingredient within the same class of pharmaceuticals for treating or preventing the same symptoms or same disease (polypharmacy), such as infectious disease, metabolic disorders, cardiovascular disease, pain, cancer, transplant-related treatment, gastrointestinal disorders, respiratory diseases, autoimmune diseases and vaccines; anti-infective active pharmaceutical ingredient comprising first and second antibiotics; an anti-viral and an anti-bacterial pharmaceutical ingredient; a pharmaceutical ingredient, for treating cancer and managing symptoms of cancer, for example topoisomerase inhibitor drug, and an anti-cancer monoclonal antibody drug, an antibiotic and an antibiotic potentiator; a fast release or fast action and slow release or long term action formulations of the same pharmaceutical, such as nitroglycerin, with fast acting/fast dissolving formulation providing for a fast action for acute treatment with a slow release formulation for maintenance; antibiotic with fast action/fast dissolution formulation for immediate increase of the concentration in blood plus slow release; pain medication, with a fast acting formulation for immediate pain relief help combined with a slow release pain maintenance medication; sleeping aid with a fast dissolving or fast acting formulation for immediate effect combined with a delayed release for maintenance throughout the night, such as Ambien; two anti-cholesterol pharmaceutical ingredients such as statins of different types combined in the combination medication delivery system; a broad spectrum anti-hypertensive combination comprising two or more hypertension-reducing drugs, including medications of the same type, such as beta-blockers or diuretics, or medications of different types or classes, such as beta-blocker and diuretic; two or more anti-malaria drugs such as Artesunate and Mefloquine; Artemether and Lumefantrine; Chloroquine and Paracetamol; and at least two of the following: Artemether; Lumefantrine; Artensunate; Amodiaquine HCl; Atovaquone-proguanil; Quinine Sulfate; Chloroquine Sulfate; Hydroxychloroquine Sulfate; Doxycycline; Mefloquine; Primaquine; Sulfadoxine; Pyrimethamine; Paracetamol; at least two nucleoside reverse transcriptase inhibitor (NRTI) medications, including e.g. Abacavir; lamivudine; Didanosine; Emtricitabine; Stavudine; Tenofovir, a non-nucleoside reverse transcriptase inhibitor (NNRTI) and a nucleoside reverse transcriptase inhibitor (NRTI) e.g. Nevirapine (NNRTI) and didanozine (NRTI); Efavirenz (NNRTI) and abacavir sulfate (NRTI); two NRTI's and one NNRTI, e.g. Abacavir and lamivudine and efavirenz or Abacavir and lamivudine and nevirapine; at least two 2 NRTI's and a PPI such as Abacavir and lamivudine and lopinavir/ritonavir; at least two anti-HIV drugs selected from the group consisting of: abacavir sulfate; didanozine; stavudine; tenofovir; disoproxil; fumarate; zidovudine; lamivudine; emtricitabine; lopinavir/ritonavir; nevirapine; efavirenz and nelfinavir; a combination of AZT and 3TC; a combination of abacavir and AZT and 3TC; a combination of lopinavir and ritonavir; combinations of ABC and 3TC; a combination of emtricitabine and tenofovir; at least two of Tuberculosis treatment medications selected from: Isoniazid; Rifampicin; Pyrazinamide; Ethambutol HCl; Streptomycin; Capreomycin; Cycloserine; Protionamide; Macrolides; Fluoroquinolones; and p-Salicylic acid; at least two of the pain treatment medications selected from: Aspirin; Carbex; Codeine; Luvox; Marplan; Nardil; Neurotin; OxyContin; Parnate; Topamax; Tylenol/Acetaminophen; Vicodin; Xyrem; Zarontin; Zoloft and Zomig; a pH buffering compound and/or an anti-acid compound in combination with aspirin; and a combination therapy for treatment of lupus nephritis, such as methylprednisolone and cyclophosphamide.

27. A pharmaceutical delivery package as claimed in claim 26, comprising fixed unit dose quantities of two or more different pharmaceutical ingredients (a) combined in a single delivery package, and (b) segregated from one another within said package, wherein the package, when split in half will contain equal amounts of the pharmaceutical ingredients.
